SEQUENCE LISTING (23 sequences)

SEQ ID NO: 1 - DNA, 13 bp, Artificial Sequence
  Description of Artificial Sequence Synthetic primer
tctgatgaataac

SEQ ID NO: 2 - DNA, 24 bp, Artificial Sequence
  Description of Artificial Sequence Synthetic primer
gaacgtgtacatcgactgaaatcc

SEQ ID NO: 3 - RNA, 10 nt, Dengue virus
acgagcguau

SEQ ID NO: 4 - RNA, 10 nt, Dengue virus
gagcgaguuc

SEQ ID NO: 5 - DNA, 96 bp, Dengue virus
acagttttaatggtcctcgtccctgcagcattccaagtgagaatctctttgtcagctgttgtacagtcgacacgcggtttctgagcgctttcagca

SEQ ID NO: 6 - DNA, 39 bp, Artificial Sequence
  Description of Artificial Sequence Synthetic primer
cgaggtacctaaaaaaaatcatgaatggcatcaactctg

SEQ ID NO: 7 - DNA, 62 bp, Artificial Sequence
  Description of Artificial Sequence Synthetic primer
tgtgcggccgcagtctagaaaaggatcctagatcataatcagccataccacatttgtagagg

SEQ ID NO: 8 - DNA, 36 bp, Artificial Sequence
  Description of Artificial Sequence Synthetic primer
aaactcgagaccatggaagacgccaaaaacataaag

SEQ ID NO: 9 - DNA, 40 bp, Artificial Sequence
  Description of Artificial Sequence Synthetic primer
tttaaatttcgtctccagacatggtcctaaccacgcagcc

SEQ ID NO: 10 - DNA, 68 bp, Artificial Sequence
  Description of Artificial Sequence Synthetic primer
aaatcttacgcgtcggtttctctatgcgagcattcggcataaaaagttatcaggcatgcacctggtag

SEQ ID NO: 11 - DNA, 84 bp, Artificial Sequence
  Description of Artificial Sequence Synthetic primer
gggaacggctgacacgcggtttctgagcgctttcagcacttgagcgaggaagggcgtaaaagttatcaggcatgcacctggtag

SEQ ID NO: 12 - DNA, 100 bp, Artificial Sequence
  Description of Artificial Sequence Synthetic primer
gggaaaacggctacagttttaatggtcctcgtccctgcagcattccaagtgagaatctctttgtcagctgttgtacagtcgacacgcggtttctgagcgc

SEQ ID NO: 13 - DNA, 33 bp, Artificial Sequence
  Description of Artificial Sequence Synthetic primer
aaaacggctatggtgagcaagggcgaggagctg

SEQ ID NO: 14 - DNA, 60 bp, Artificial Sequence
  Description of Artificial Sequence Synthetic primer
aaagggaaagcgcgctaatacgactcactatagggagtctacgtggaccgacaaagacag

SEQ ID NO: 15 - DNA, 38 bp, Artificial Sequence
  Description of Artificial Sequence Synthetic primer
tttccctttctcgaggactagcggttagaggagacccc

SEQ ID NO: 16 - DNA, 42 bp, Artificial Sequence
  Description of Artificial Sequence Synthetic primer
cccctcgaggattcttctgacacaacagtctcgaacttaagc

SEQ ID NO: 17 - DNA, 62 bp, Artificial Sequence; identical to SEQ ID NO: 7
  Description of Artificial Sequence Synthetic primer

SEQ ID NO: 18 - DNA, 47 bp, Artificial Sequence
  Description of Artificial Sequence Synthetic primer
cccgggcccgcggccgcttttcttgacttctcttaaaaccaagaatg

SEQ ID NO: 19 - RNA, 46 nt, Dengue virus
gaccaccuuucaauaugcugaaacgcgagagaaaccgcguaucaac

SEQ ID NO: 20 - RNA, 111 nt, Dengue virus
acacgccuuucaauaugcugaaacgcgagagaaaccgcgugucgacuguacaacagcugacaaagagauucucacuuggaaugcugcagggacgaggaccauuaaaacugu

SEQ ID NO: 21 - RNA, 41 nt, Dengue virus
acgccuuucaauaugcugaaacgcgagagaaaccgcguguc

SEQ ID NO: 22 - RNA, 47 nt, Artificial Sequence
  Description of Artificial Sequence Synthetic oligonucleotide
gacacgcgguuucugagcgcuuucagcacuugagcgaggaagggcgu

SEQ ID NO: 23 - RNA, 18 nt, Artificial Sequence
  Description of Artificial Sequence Synthetic oligonucleotide
uaaucgauccucgagacc

(19) United States
(12) Patent Application Publication
(10) Pub. No.: US 2012/0278913 A1
(43) Pub. Date: Nov. 1, 2012

(54) Ribozyme Effector Gene in Dengue Fever Transmission and Disease Control

(75) Inventor: Malcolm J. Fraser, Granger, IN (US)

(73) Assignee: University of Notre Dame, Notre Dame, IN (US)

(21) Appl. No.: 13/096,649

(22) Filed: Apr. 28, 2011

(51) Int. Cl. A01K 67/027; C12N 5/10; C12N 15/63

(52) U.S. Cl. 800/21; 435/320.1; 435/348

(57) ABSTRACT

Disclosed are anto-DENV ribozyme based methods and compositions useful in the inhibition and control of all Dengue fever serotypes (designated DENV 1 through 4). A group of anti-DENV Group 1 trans-splicing introns (αDENV-GrpIa) are presented that target DENV-2 NGC genomes in situ. Methods for specifically targeting a highly conserved 5′-3′ cyclization sequence (CS) region that is common to all serotypes of the DENV are provided. The anti-DENV Group 1 trans-splicing introns (αDENV-GrpIa) specifically target two different uracil bases on the positive sense genomic strand. The invention provides an RNA based approach for transgeneic suppression of DENV in transformed mosquitoes using a group of specifically designed introns that trans-splice a new RNA sequence downstream of a targeted site. The aDENV-GrpIs target DENV infected genomes and thus provide a method for inhibiting the spread of Dengue fever. An αDENV-GrpI 9v1 is presented that is designed to be active against all forms of Dengue virus, and to effectively target the DENV-2 NGC genome in a sequence specific manner

### STATEMENT OF GOVERNMENT SUPPORT

The United States Government may own rights in the present invention as support for these studies was provided by NIH/NIAD RO1A1048561.

### BACKGROUND

1. Field of the Invention

The present invention relates generally to the field of disease control, and particularly to the control of the transmission and infection of Dengue fever. More particularly, the present invention relates to a system and method for controlling and or inhibiting the transmission of Dengue fever by mosquitoes through the use of anti-Dengue virus trans-splicing group I introns.

1. Background of the Invention

Like other Flaviviruses, Dengue virus (DENV) enter the cell by receptor mediated endocytosis (REM)^{35,36}. Following acidification of the endosome and membrane fusion the 9.6 kb positive-sensed DENV genome is released into the cytoplasm where replication begins. This is an ideal place for a trans-splicing ribozyme to attack the DENV genome. One strategy currently under development to directly attack the Dengue genome is the use of the RNAi response in mosquitoes³⁷⁻⁴². The wild-type RNAi response mounted by the mosquito itself in reaction to an infection may not be strong enough to protect against the spread of the virus, or Dengue would cease to be a concern. However, pre-priming mosquito cells for RNAi protection against Dengue through the expression of Dengue-specific dsRNA before any infection occurs is an effective approach, severely hindering replication of the virus in some cases³⁹. This tactic suffers from the same drawbacks as the vaccine: escape mutants.

The error rate of the Dengue RNA polymerase suggests that, on average, one random mutation arises for every replication event. Previous work has shown limited mismatching of the RISC complex RNA to its target is tolerated to a degree¹¹. In contrast, the ability of an induced RNAi response to discriminate between different alleles of the same gene varying only by a single nucleotide has also been observed⁴³. Whether or not a particular mutation within the targeted region confers resistance appears to depend on the location of the altered base, as well as the nature of the alteration itself. Selective pressure within the cells of a mosquito may initially silence a Dengue infection, but eventually that pressure would serve to promote the replication of virus genomes carrying mutations conferring resistance to the RISC complex nuclease activity. Such mutants would be transmitted by the insect and spread throughout a population even in the presence of the protective measures granted by RNAi, eventually rendering the specific sequence utilized in priming a mosquito for an RNAi response useless in the face of the escape mutant strain of Dengue.

Dengue viruses (DENV) are one of the most important viral diseases in the world with approximately 100 million infections and 200,000 deaths each year. The current lack of an approved tetravalent vaccine and ineffective insecticide control measures warrant a search for alternatives to effectively combat DENV. The trans-splicing variant of the Tetrahymena thermophila group I intron catalytic RNA, or ribozyme, is a powerful tool for post-transcriptional RNA modification. The nature of the ribozyme and the predictability with which it can be directed makes it a powerful tool for modifying RNA in nearly any cell type without the need for genome-altering gene therapy techniques or dependence on native cofactors.

The mosquito-borne Dengue viruses (DENV) are responsible for approximately 100 million infections and 200,000 deaths each year with 2.5 billion people remaining at risk for DENV infection, making DENV one of the most important viral diseases in the world (1). Infection with one of four antigenically distinct, but related Dengue virus serotypes (designated DENV 1 through 4) can result in Dengue fever (DF) and/or Dengue hemorrhagic fever (DHF)¹. DF and DHF are endemic to tropical and subtropical regions of the world, but global changes in climate, rapid dispersal of virus due to ease of global travel, and migration of humans to non-tropical regions has resulted in DENV outbreaks in areas that were once non-endemic to the Dengue viruses^{2,3}. Modem travel and shipping inevitably leads to an increase in the number of cases in developed countries as well, including a recent outbreak in the Hawaiian islands in 2001 (Source: CDC). These viruses are maintained in a cycle that involves humans as well as the dipteran Aedes aegypti mosquito which preferentially feeds on human blood and is widely distributed throughout the world^{2,3}.

The current lack of an approved effective tetravalent vaccine and the ineffectiveness of insecticide control measures continue to warrant a search for alternative strategies to effectively combat DENV. Newer approaches that have received considerable attention include interference with the extrinsic incubation cycle of DENV replication within the arthropod vector^{2,3}. One such approach envisions population replacement of vector competent mosquitoes with those refractory for infection and/or transmission of the virus, which could theoretically halt disease transmission^{2,3}. This approach has distinct advantages for environmental safety, cost effectiveness, and long term disease suppression.

The trans-splicing reaction of the Group I intron is derived from the natural cis-splicing reaction. Both the cis and trans-splicing reaction can be divided into two distinct successive transesterification steps⁵. The primary difference between the two reactions is that while the cis-splicing reaction occurs along one continuous RNA molecule to join a 5′ and a 3′ exon, the trans-splicing intron is located on the same molecule as the 3′ exon, but seeks out a separate 5′ exon to which it can append the 3′ exon⁶.

The engineered trans-splicing activity of the Group I intron is a versatile tool with respect to the ‘editing’ of RNA⁷⁻¹⁷. Group I intron trans-splicing has been used in repair of mutant α-globin mRNA⁸, restoration of wild-type p53 activity in three cancerous cell lines¹⁸, re-establishment of the function of the canine skeletal muscle chloride channel¹⁹, and induction of p16 activity in a pancreatic cell line¹⁰, and trans-splicing group-I intron targeting of the HIV-1 tat²⁰, cucumber mosaic virus coat protein mRNAs⁷, and the hepatitis C virus internal ribosome entry site (HCV-IRES)²¹.

Group I introns are subject to the same limitations as antisense or RNAi methods of RNA suppression because the high mutation rate of the DENV genome promotes the spread of strains capable of avoiding the antisense recognition essential to the trans-splicing reaction. Approaches that inhibit DENV infection by direct interaction with the RNA genome must be designed to act upon invariant sequences to be effective. The most invariant segments of the DENV genome are the 5′ and the two 3′ cyclization sequences (5′CS, CS1, and CS2 respectively) which are involved in the formation of a panhandle structure that is apparently essential for genome replication^{22,23}. These cyclization sequences are separated by such a large intervening length of RNA that they are effectively acting in a trans manner, and since they are able to base-pair with each other their secondary structure is likely open and conducive to base-pairing.

The 5′CS is located downstream of the polyprotein start codon, well within the ORF of the Capsid (CA) protein. The stringency of tolerable mutations in this sequence may be increased by the need of the virus to conserve a functional CA protein. In fact, all mosquito-borne flaviviruses share an 8 by stretch of nucleotides within this 5′ CS sequence²⁴.

### SUMMARY OF THE INVENTION

The present invention provides an RNA based method, specifically using a DENV ribozyme strategy, for intracellular suppression of virus infection as a means of transgenic immunization of mosquitoes. The ribozymes presented have targeted sequences that are conserved among all Dengue virus serotypes. In some embodiments, the conserved Dengue virus sequence targeted using the present RNA based methods is a cyclization sequence (CS) of the Dengue virus genome, and particularly a 5′ and the two 3′ cyclization sequences (5′ CS. CS1 and CS2, respectively) of the native (wild-type) Dengue virus genome. The 5′ CS is located downstream of the polyprotein start codon, and well within the ORF of the Capsid (CA) protein.

In one aspect, the invention provides Group I introns having a trans-splicing activity. These introns are employed as part of a method for inhibiting insect (e.g., mosquito) transmission of Dengue virus infection to an animal In some embodiments, the intron may be described as having an internal guide sequence (IGS) (a part of the PI helix) and an external guide sequence (EGS), each of which are complementary to the target RNA sequence of the Dengue virus. In some embodiments, the IGS is limited in size to 9 base pairs near a designated reactive uracil residue (U). In other embodiments, the EGS may be described as having virtually any length, and as being capable of forming a transient helix with a target RNA sequence of the Dengue virus that is located downstream of the designated reactive uracil residue.

In another aspect, a method is provided for inhibiting Dengue virus transmission comprising exposing a population of insect (mosquito) cells or insects (mosquitoes) to a ribozyme effector gene comprising an anti-Dengue virus trans-splicing intron that targets a highly conserved cyclization sequence (CS) within the DENV genome. In some embodiments, the intron is designed to target a sequence target sequence within the DENV genome that is further defined as a uracil residue located within a native (wild-type) Dengue virus conserved cyclization encoding sequence. In some embodiments, the targeted conserved sequence may be described as a sequence from nucleotide base pair C131 to nucleotide base pair C151 of the native DENV genome, with a particular target being identified as a uracil target nucleotide within this sequence that corresponds to position U143, U132, or both, as they are identified by position relative to the native Dengue virus sequence nucleotide positions within the CS encoding sequence. The native sequence encoding for the CS in the Dengue virus genome is described herein relative to the native (wild-type) DENV-2 New Guinea strain C genome (DENV-2 NGC; GenBank Accession: M29095).

In another aspect, anti-DENV Group trans-splicing introns (αDENV-GrpIs) are presented, these introns being capable of targeting DENV-2 NGC genomes. These introns may be further described as targeting specific uracil bases within the positive sense genomic strand within the highly conserved 5′-3′ cyclization sequence (CS) region that is conserved within all serotypes of the Dengue virus. (for example, serotypes 1, 2, 3 and 4). In particular embodiment, the intron is designated αDENV-Grp1 9v1 or αDENV-Grp1 96v4.

In another aspect, the invention proves a method for targeting an infecting Dengue virus genome in an insect. In some embodiments, the insect is a mosquito, and in particular, an Aedes aegypti mosquito.

In yet another aspect, a transfected or transformed insect and insect cell line are provided, the insect and/or insect cell line being transfected with the anti-DENV Group 1 trans-splicing introns described herein, and being resistant to infection by Dengue virus.

Other objects and advantages of the present invention will become apparent to those skilled in the art upon reading the following detailed description of preferred embodiments.

### BRIEF DESCRIPTION OF THE DRAWINGS

FIG. 1 relates to αDENV-GrpI targeting and trans-splicing of DENV. Schematic diagrams showing DENV sequences targeted by 9 series (A) and 96 series (B) αDENV-GrpIs (shown as blow-ups), and the trans-splicing reaction mediated by these introns (C) as previously described for other trans-splicing ribozymes (5). A) A region of the DENV genome targeted by the 9 series intron is conserved among all serotypes based on Clustal X DENV genome alignment of all 98 DENV sequences deposited at GenBank. All accession numbers are listed in Methods. Shaded areas indicate 100% conserved bases. The arrows designate U residues targeted for cleavage. The residues contributing to structural elements of the intron RNA-target RNA complexes in the different 9 series introns are as follows: Internal Guide Sequence (IGS)=U138 to A146, Bulge Loop (BL)=A147 to A 152, and External Guide Sequence (EGS)=G153 to G161 as indicated on. Uracil 143 is targeted for cleavage. The single adenine at base 152 is not fully conserved and has therefore been engineered into LB, which is not required to base pair with the target for efficient splicing. B) Primary sequence of the DENV 2-NGC genome depicting the residues contributing to structural elements of the intron RNA-target RNA complexes in the different 96 series intron structures: IGS=A127 to C135, LB=A136 to A 139, and EGS=U140 to U 235. The uracil at position 132 is targeted for cleavage. C) The αDENV-GrpI-mediated trans-splicing reaction. The DENV target sequence is represented as the upper schematic diagram with the trans-splicing ribozymes shown below. See text for description. Figure not drawn to scale.

FIG. 2 relates to Anatomy of trans-splicing group I introns. The IGS forms the P1 helix with the target to bring it into proximity with the GNP 3′-OH. The EGS is an extra stretch of antisense RNA that improves the efficiency and specificity of intron targeting the desired viral sequence. The formation of the P10 helix brings the newly cleaved 3′-OH of the uracil into proximity with the 3′ exon, allowing covalent splicing. P1 and P10 overlap by 2 by IGS upon targeting of the DENV-2 genome. A UAA stop codon was inserted in the trans-splicing domain of each intron immediately upstream of the UCG splice site to prevent inadvertent translation of the 3′ exon until formation of the DENV 2-FL spliced product. The sites of trans-esterification on the phosphodiester backbone (1 and 2) are marked with black arrows.

FIG. 3 relates to Quantitative luciferase analysis of αDENV-GrpIs splicing efficiency. A) Schematic diagram of the αDENV-GrpI-FL constructs used for this analysis. A5c=Drosophila melanogaster actin 5c promoter, FL=firefly luciferase. B) Drosophila S2 cells were co-transfected with 3 μg αDENV-GrpIs, 0.5 μg of the IRL expression plasmid, and 1 μg of either double-stranded DENV-2 target (+) or control plasmid, pUC57 (−) (see Methods). Following cell lysis, samples were processed using the Dual Luciferase System (Promega) according to the manufacturer's direction. The error bars represent standard deviations of three independent experiments.

FIG. 4 relates to Engineering and fluorescence microscopy of αDENV-GrpIs in a Bicistronic Plasmid. A) Each of the trans-splicing αDENV-GrpI was tagged downstream of the firefly (FL) 3′ exon with the mCherry fluorescent marker gene expressed from an IRES sequence of either the Black Queen Cell Virus (BQCV) or Drosophila C Virus (DCV). Expression of these constructs was driven by the Drosophila melanogaster Actin 5c promoter. This bi-cistronic configuration allowed monitoring for the presence and expression of the αDENV-GrpI constructs within cell cultures. A5c=Drosophila melanogaster actin 5c promoter, IRES=DCV or BQCV Internal ribosome entry site. B) Expression of mCherry was verified for each construct by transfecting 1 μg of plasmid DNA into C6/36 cells and examining at 48 hours post transfection. Photographs were taken under 40× magnification.

FIG. 5 relates to Assessment of αDENV-GrpI bicistronic constructs activities using luciferase assays. A) Drosophila S2 cells were co-transfected with 3 μg of each αDENV-GrpI, 1 μg double-stranded DENV-2 target (+) or control plasmid (−), and 0.5 μg of IRL expression plasmids. Following cell lysis, samples were processed and assayed for luciferase activity (see Methods). The error bars represent standard deviations of three independent experiments. B) Aag2 cells co-transfected with αDENV-GrpI and pA5c-IRL expression plasmids were challenged with DENV-2 NGC at an MOI of 0.01 24 h post transfection (+). Control cells were transfected with an empty pUC57 plasmid (−) and challenged with virus in the same manner. The error bars represent standard deviations of three independent experiments. B=BQCV IRES. D=DCV IRES. RL=Renilla luciferase. RLU=Relative Luciferase Units.

FIG. 6 relates to αDENV-GrpI constructs effectively target DENV-2 NGC. T-25 flasks containing D. melanogaster S2 cells (5×105) transiently transfected (A) or transformed (B) with αDENV-GrpI bicistronic constructs, were transfected with double-stranded DENV-2 target (+), or the negative control pUC57 empty vector (−). Resulting RNAs were analyzed by RT-PCR in the presence (+Rt) or absence (−Rt) of reverse transcriptase to insure that observed amplified products were derived from RNA. A PCR amplification product derived from a constructed spliced sequence control (Methods) is provided as size standard for each gel (DNA+Ctrl). Arrows indicate the predicted size of the principle splice products resulting from intron activity. The identity of splice product was confirmed by sequencing.

FIG. 7 relates to αDENV-GrpI-FL constructs effectively target and suppress DENV-2 in mosquito cells. A) Confirmation of representative αDENV-GrpI trans-splicing activities. Ae. Albopictus C6/36 cells were transiently transfected with trans-splicing αDENV-GrpI bicistronic vector constructs. Resulting RNAs were analyzed by RT-PCR in the presence (+Rt) or absence (−Rt) of reverse transcriptase to insure observed amplified products were derived from RNA. A PCR amplification product derived from a constructed spliced sequence control (Methods) is provided as a size standard for each gel (DNA+Ctrl). Δ9 and Δ96 refer to Pabc5 deletion mutations located in the trans-splicing domain of the group I intron. These deletions are designed to knock out function providing an adequate negative control⁵². Arrows indicate the predicted size of the principle splice products resulting from intron activity. The identity of spliced product was confirmed by sequencing. B) C6/36 cells were transformed with trans-splicing αDENV-GrpI bicistronic vector constructs and maintained under 10 mg/ml hygromycin selection for more than 30 doublings. Transformed cells were washed twice in serum free media at 15 hours post plating, infected with DENV-2 NGC (MOI=0.1). RNAs were analyzed by RT-PCR as described in A). Arrows indicate the predicted size of the principle splice products. C) Ae. albopictus C6/36 cells were transformed with group I intron vector constructs and maintained under hygromycin selection. Transformed cells were washed three times and challenged with DENV 2-NGC (MOI 0.01). Infections were allowed to proceed for 4 days, supernatants were collected, and viral titers were determined by TCID50-IFA as described herein.

FIG. 8: RT-PCR analysis of mosquito cells expressing αDENV-Grp1 following challenge with DENV. Primers used were specific for splice product. Left: Mosquito cells transiently transfected with plasmid expressing αDENV-Grp1 and challenged with DENV. Right: Transformed mosquito cells constitutively expressing αDENV-Grp1 introns challenged with DENV.

FIG. 9: Suppression of DENV infections in mosquito cell cultures transformed with a αDENV-Grp1 introns. Two targeting sequences (indicated by the 9 or 96 designation) were used to effect cleavage within the conserved CS sequence found among all DENV genomes. The titers for infected (I) and uninfected (u) untransformed cells are shown as the bars 1 and 2 from the left, respectively. The 9 version αDENV-Grp1-tBax introns 9tB, 9tB-B, and 9tB-D (bars 3, 4, and 5, respectively) were designed to target all DENV serotypes and strains, while the 96 version αDENV-Grp1-tBax introns 96tB, 96tB-B, and 96tB-D (bars 6, 7, and 8, respectively) were designed to target an extended sequence in DEN2-NGC. Both intron designs were capable of effectively suppressing the DENV-1 Hawaii serotype, with the 9 version constructs exhibiting the greatest effectiveness. Similar results were obtained for DENV serotypes 2, 3, and 4. Background virus growth is believed due to the inefficiency of transformation and hygromycin selection of transformed cells.

FIG. 10: RT-PCR amplification of splice products of in vitro trans-splicing reaction against both single stranded and double stranded synthetic RNA targets.

### DETAILED DESCRIPTION OF THE PREFERRED EMBODIMENTS

The studies presented here are not conducted in a way that one can assess if the Grp-1 approach is superior or inferior to siRNA/shRNAs. siRNA does not carry out splicing. In the present invention, Grp1 introns have been specially designed to target and splice a specifically identified multiple serotype conserved DENV capsid sequences. Further, an abundance of siRNA work has already been performed with DENV^{44,45}, and the conserved sequence subject as part of the present invention is a sequence that is not among those identified as useful for targeting. Among other reasons, it is observed that the length of the conservation for this sequence employed in the present compositions and methods among all DENV is smaller than the sequence length that is reported to be required for an siRNA response.

Group I trans-splicing introns have a demonstrated potential for targeting RNA virus genomes in infected cells [20,21]. The feasibility of using αDENV-Grp1s to catalyze trans-splicing of the 5′ conserved region of the DENV family genomes is herein demonstrated. In designing Group I intron splicing approaches, most studies employ a GN5 library scan to map those uracils most accessible to trans-splicing in an otherwise highly invariant sequence [28]. The highly mutable nature of the Dengue genome, however, precludes this approach, as any uracils identified may or may not be present in other serotypes or even other strains of the same serotype.

In addition to the questionable presence of the uracil, the immediate neighboring sequence must also be conserved to facilitate targeting through base pairing interactions. The optimal Group I intron target following alignment of 98 instances of DENV from GenBank which identified one conserved region that appeared to satisfy the requirements for trans-splicing within the DENV genome. This region is positioned within the CS (or CA) encoding sequence of the Dengue virus genome, specifically a sequence located at nucleotide positions C131 to G151. The native Dengue virus genome cyclization sequence is described in Alvarez et al. (2005) 23 and Alvarez et al. (2008) 25, both of these being specifically incorporated herein by reference. This sequence region contains a number of possible uracil targets for the trans-splicing reaction. This region is a part of a double-stranded 5′-3′ CS domain that forms as a result of complementary base pairing between the proximal ends of the 5′ and 3′ UTRs during DENV replication [23]. The formation of the 5-3′ CS domain has been shown to be essential for DENV replication [25]. An anti-DENV Group I trans-splicing conserved region sequence that includes a uracil residue corresponding to a native gene sequence at position 143 (U143) and a uracil residue located at a native gene sequence uracil residue at position 123 (U123), was designed as part of the present invention.

Intron 9v1, which has a 9 base P1 helix, and a 9 base EGS, is designed to effectively trans-splice all known DENV sequences. This intron demonstrated an ability to cleave at U143 and effectively trans-splice an infecting DENV 2 NGC genome either upon transfection of Aag2 cells or as a constitutively expressed RNA in transformed C6/36 cells.

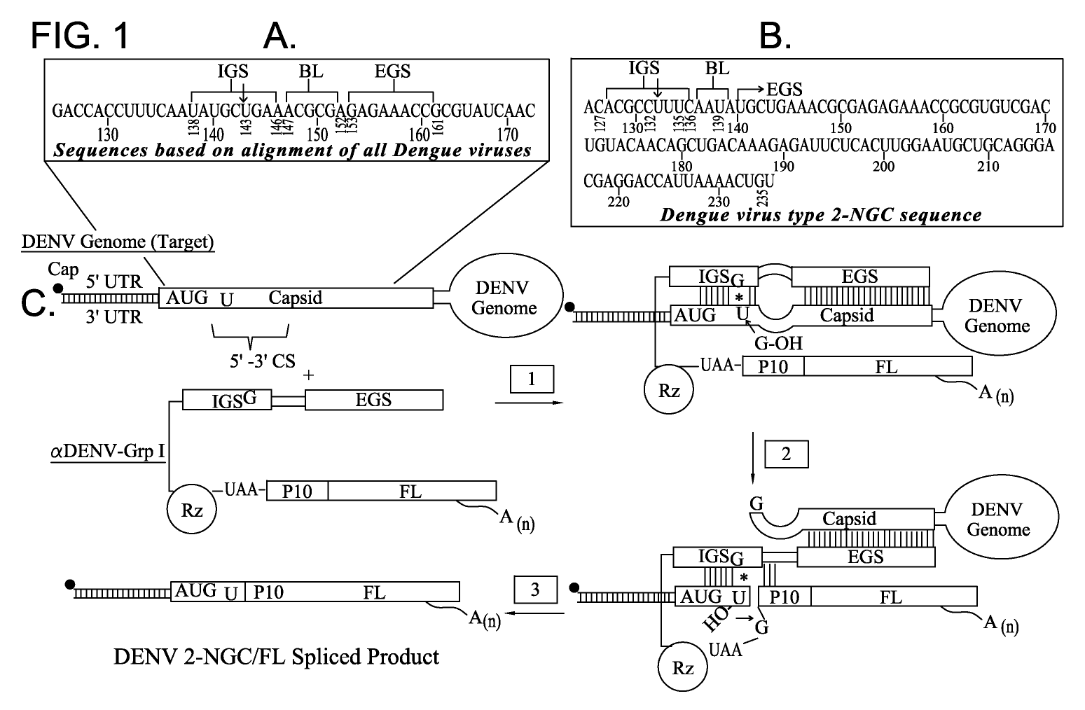

A separate set of αDENV-Grp1s were constructed with an extended 96 base antisense EGS that was engineered to target the DENV-2 NGC (FIG. 1b). Each version of this series shares the same EGS and P1 helix, and targets U123, but differs in their P10 helix. 96v1 has a 6 base pair P10 helix with no wobble base, and a standard P1 helix including the required wobble base. 96v3 differs from 96v1 in trimming of 3 nucleotides between the P10 helix and the catalytic core, while 96v4 incorporates a wobble base pairing downstream of the 3′ exon splice-site.

Each αDENV-Grp1 was constructed with a 3′ firefly luciferase (FL) ORF that permitted quantitative assessment of splicing activity. Co-transfection assays for FL activity were performed in S2 or Aag2 cells using the fold back DENV-2 mimic plasmid and either BQCV or DCV IRES/mCherry-linked αDENV-Grp1 9v1 or 96v4 expression plasmids introns. Although all introns assayed exhibited firefly luciferase activity, and therefore the greatest amount of trans-spliced product. This is likely due to both the relative activity of the intron configuration as well as an increase efficiency of targeting as a result of the extended EGS. The αDENV-Grp1 as 96v1, designed to target all DENV serotypes, also displayed a significant ability to successfully splice our DENV mimic in these cells, but its reduced level of FL activity reflects a somewhat reduced efficiency of targeting relative to αDENV-Grpl as 96v4. This is likely due to the presence of a shorter EGS, as this has been previously shown to decrease the ability of a trans-splicing intron to attack a target sequence [7]. Alternatively, the relative effectiveness of cleavage for U143 targeted by the 9v1 intron may be less than that for U132 targeted by the 96v4 introns. Nonetheless, this intron was still quite effective in targeting and splicing the DENV sequence.

Similar results were obtained with Aag2 cells transfected with these αDENV-Grp1 constructs were challenged by infection with DENV-2 NGC. These results validated the potential of the present αDENV-Grp1 intron approach as an effective means of suppressing DENV infection of mosquito cells and tissue.

The addition of a 3′ IRES/mCherry configuration, whether incorporating the BQCV IRES or the DCV IRES, did not appear to alter the trans-splicing capabilities of either intron, because the IRES allows expression of the mCHerry fluorescence marker in the unspliced intron. In some embodiments, this may be used to provide a convenient independent marker for determining the relative efficiency of expression of the introns following transfection.

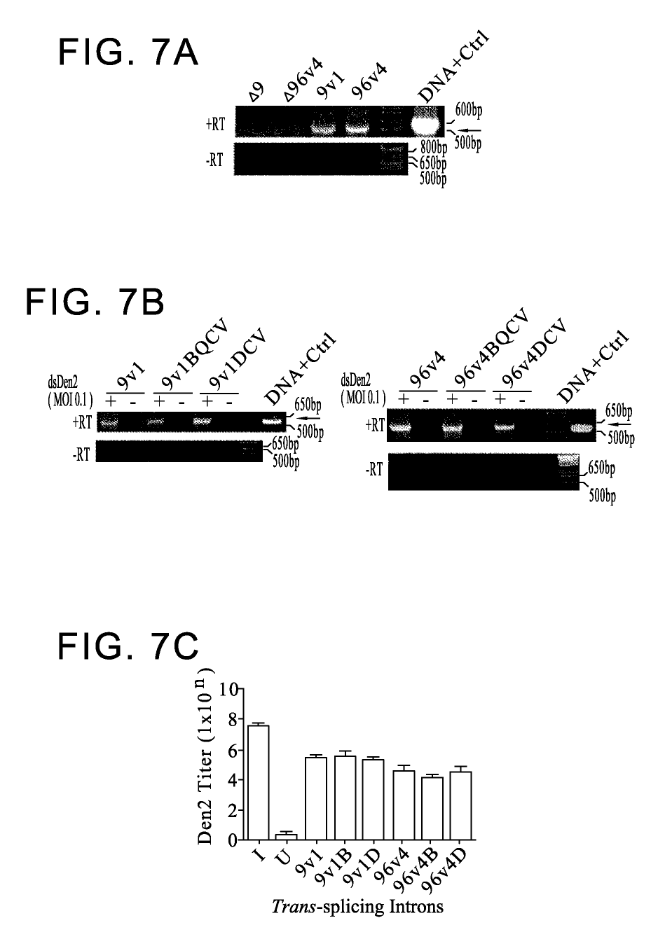

The use of these intron constructs to function in transformed mosquito tissues was confirmed by demonstrating their activity against infectious DENV-2 NGC in transformed C6/36 cells expressing the bicistronic αDENV-Grp1 9v1 or 96v4, either linked with the BQCV or DCV IRES driven mCherry, or lacking an IRES mCHerry linkage (FIG. 7). RT-PCR amplified DENV-2-FL splice product was obtained with αDENV-Grp1 9v1 compared to that resulting from αDENV-Grp1 96v4 activity, and may once again be due to differences in the lengths EGS of these two introns since EGS length is a determining factor for Grp1 trans-splicing efficiency [7].

Further validation of these introns as tools to combat DENV is evidenced to TCID₅₀-IFA analyses that test suppression of overall infectious virus production (FIG. 7). A 2 log (for 9v1) to 3 log (for 96v4) was observed demonstrating these αDENV-Grp1 do suppress DENV 2NGC infection of the transformed cells, and the αDENV-Grp1 9v1, designed to target all four Dengue serotypes, has the ability to suppress the replication of all serotypes of this virus.

The results presented here show that 9v1 intron, designed to be active against all forms of Dengue virus, is capable of effectively targeting the DENV 2-NGC genome in a sequence specific manner, while suppressing virus production. These novel αDENV-Grp1s provide an attractive alternative to other RNA based approaches for the transgenic suppression of DENV in transformed mosquito cells and tissues.

Definitions:

The following definitions are employed throughout the description of the present invention:

In the sense of the present invention, the term “identical” relates to the degree of sequence identity of a nucleic acid sequence compared to another nucleic acid sequence. Identical nucleic acid sequences, in the sense of the present invention have a sequence identity of at least 40%, at least 50%, at least 60%, preferably at least 70%, especially preferably at least 80%, also especially preferably at least 90%, in particular preferably at least 95% and most preferably at least 98 or 100% compared to another nucleic acid sequence.

The term “complementary” means the ability of a nucleic acid sequence to hybridize with another nucleic acid sequence due to hydrogen bridges between complementary bases. The skilled person knows that two nucleic acid molecules do not need to have a 100% complementarity in order to hybridize with each other. Preferably, a nucleic acid sequence, which is to hybridizes with another nucleic acid sequence, is least 40%, at least 50%, at least 60%, at least 70%, especially preferably at least 80%, also especially preferably at least 90%, in particular preferably to at least 95% and most preferably at least 98 or 100% complementary to said nucleic acid sequence.

According to the invention, the “recombinant nucleic acid molecule” stands for all vectors, plasmids, cosmids, viruses and other vectors common in genetic engineering, for the transfer/introduction of nucleic acid molecules in insects or insect cells.

The nucleic acid sequence of the present invention may consist of or be derived from a naturally occurring nucleic acid sequence or a synthetically produced, by sequence comparison derived or recombinantly produced nucleic acid sequence.

The term “hybridizing under stringent conditions” denotes in the context of the present invention that the hybridization is implemented in vitro under conditions which are stringent enough to ensure a specific hybridization. Stringent in vitro hybridization conditions are known to those skilled in the art and may be taken from the literature (e.g. Sambrook and Russell (2001) Molecular Cloning: A Laboratory Manual, 3rd edition, Cold Spring Harbour Laboratory Press, Cold Spring Harbour, N.Y.). The term “specific hybridization” refers to the circumstance that a molecule, under stringent conditions, preferably binds to a certain nucleic acid sequence, i.e. the target sequence, if the same is part of a complex mixture of, e.g. DNA or RNA molecules, but does not, or at least very rarely, bind to other sequences.

Stringent conditions depend on the circumstances. Longer sequences hybridize specifically at higher temperatures. In general, stringent conditions are chosen such that the hybridization temperature is about 5° C. below the melting point (T.sub.m) of the specific sequence at a defined ionic strength and at a defined pH value. T.sub.m is the temperature (at a defined pH value, a defined ionic strength and a defined nucleic acid concentration), at which 50% of the molecules complementary to the target sequence hybridize to the target sequence in the state of equilibrium. Typically, stringent conditions are conditions, where the salt concentration has a sodium ion concentration (or concentration of a different salt) of at least about 0.01 to 1.0 M at a pH value between 7.0 and 8.3, and the temperature is at least 30° C. for small molecules (i.e. 10 to 50 nucleotides, for example). In addition, stringent conditions may include the addition of substances, such as, e.g., formamide which destabilizes the hybrids. At hybridization under stringent conditions, as used herein, normally nucleotide sequences which are at least 60% homologous to each other hybridize to each other. Preferably, said stringent conditions are chosen such that sequences which are about 65%, preferably at least about 70%, and especially preferably at least about 75% or higher homologous to each other, normally remain hybridized to each other. A preferred non-restrictive example of stringent hybridization conditions is hybridizations in 6× sodium chloride/sodium citrate (SSC) at about 45° C., followed by one or more washing steps in 0.2.times.SSC, 0.1% SDS at 50 to 65° C. The temperature fluctuates, e.g. under standard hybridization conditions depending on the type of the nucleic acid, between 42° C. and 58° C. in aqueous buffer having a concentration of 0.1 to 5×SSC (pH value 7.2).

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

The term “recombinant” as used herein in relation to a polynucleotide intends a polynucleotide of semisynthetic, or synthetic origin, or encoded by cDNA or genomic DNA (“gDNA”) such that it is not entirely associated with all or a portion of a polynucleotide with which it is associated in nature.

Unless defined otherwise, all technical and scientific terms used herein have the same meaning as commonly understood by one of ordinary skill in the art. Practitioners are particularly directed to Current Protocols in Molecular Biology (Ansubel) for definitions and terms of the art. Abbreviations for amino acid residues are the standard 3-letter and/or 1-letter codes used in the art to refer to one of the 20 common L-amino acids.

### EXAMPLE 1

### Alignment of all DENV Genomes

DENV sequence data was obtained from the National Center of Biotechnology Information (NCBI). Sequences representative of all four serotypes of Dengue were aligned using ClustalX⁴⁶. The aligned sequences comprise the following GenBank GenInfo identifiers:

12018173, 12018169, 12018171, 12659201, 2909798, 2909788, 2909786, 2909796, 6841603, 6841595, 6841605, 6841591, 6841601, 6841597, 6841593, 6841599, 6841587, 6841585, 6841589, 1000740, 1000738, 2909784, 1000736, 4926937, 4926935, 4926927, 4926929, 4926931, 2909794, 2909792, 1000742, 4926933, 2155257, 2723944, 323447, 6581076, 6581078, 2723942, 323449, 323650, 18644123, 1864412, 11119731, 19744844, 18644125, 18644127, 18643733, 4337012, 13386495, 1881708, 19071809, 13926152, 9280544, 14585842, 4926947, 4926939, 323654, 4926945, 4926943, 7329983, 7329981, 13540386, 14328931, 14485523, 323660, 17129645, 22901065, 22901063, 22901061, 1854040, 1854038, 1854036, 17129647, 24417519, 24417517, 24417515, 27656962, 24417513, 19071807, 14195698, 8927332, 14328929, 12711599, 323468, 25992053, 25992047, 25992041, 25992029, 25992025, 25992055, 25992033, 19071811, 25992043, 25992039, 25992037, 25992051, 25992031, and 25992057.

Cells, Virus and Antibody. The Drosophila melanogaster In S2 cells, obtained from ATCC, were maintained in Schneider's Modified Drosophila media (Invitrogen/Gibco) 10% FBS (Atlanta Biologicals), penicillin G (100 U/ml; Invitrogen/Gibco) and streptomycin (100 μU/ml; Invitrogen/Gibco). Aag2 Aedes aegypti mosquito cells (a kind gift from Dr. Ken Olson, Colorado State University, Fort Collins, Colo.) were maintained in Schneider's Modified Drosophila media (Lonza Group Ltd., Walkersville, Md., USA) supplemented with 10% FBS, 2 mM glutamine, penicillin G and streptomycin as described for the S2 cells used in this study. Both cell types were maintained in a 28° C./5% CO2 environment.

The Dengue 2 prototype virus New Guinea C strain (DENV2-NGC) was used in this study. Viral stocks were prepared as follows. Aag2 cells were infected with DENV 2-NGC. At 7 days post-infection, cells were scraped and freeze-thawed for three (3) cycles. Cell debris was removed by spinning cell suspensions at 10000 RPM for 30 min, and aliquots of 100 ul were stored at −80° C. until used, one aliquot was used for determining the TCID 50 of the stock. This virus stock (TCID50 =10 7) was used in subsequent experiments.

Design and assembly of anti-Dengue virus group I intron constructs. All primer sequences are illustrated in Additional file 2. All PCR products described here were band isolated using either the QlAquick Gel Extraction kit (Qiagen), or the Wizard SV Gel and PCR Cleanup Kit (Promega) and used as templates for a second round of PCR with the same primers used for the initial PCR reactions and Platinum Pfx high-fidelity DNA polymerase according to the manufacturer's protocols in order to minimize the amount of contaminating circular plasmid. Following band isolation all PCR products were digested with restriction endonucleases, obtained from New England Biolabs (NEB). All vectors were Antarctic phosphatase treated (NEB) prior to ligation. The final constructs produced were sequenced and restriction digested to verify plasmid integrity and presence of the inserts.

pA5c backbone. The αDENV-GrpI were cloned into an expression vector under the control of the distal Drosophila melanogaster actin5c. The actin5c promoter was PCR amplified from the plasmid pHermes [Actin5c:EGFP]⁴⁷. Following band isolation the PCR product and the vector pBlueScriptll SK+(Stratagene, La Jolla, Calif.) were band isolated and digested with the restriction enzymes Acc65I and NotI, and ligated using T4 DNA ligase (NEB) to give the plasmid pBlueScriptll SK+Actin5c (pBSII-A5c). The SV40 late transcription terminator and polyadenylation signal was amplified from the plasmid pMT/V5-HisA (Invitrogen). The SV40 PCR product and pBSII-A5c were digested with NotI and SadcI (NEB). The plasmid and insert were ligated together with T4 DNA ligase to yield the vector pA5c, and served as the backbone for the rest of the plasmids produced unless otherwise noted.

pA5c-FL. The firefly luciferase open reading frame was PCR amplified from the vector pGL-Basic (Promega). The PCR product and pA5c vector were a digested with XhoI and NotI. and ligated to yield pA5c-FL.

Group I introns. All introns were derived from the catalytic core of the rRNA Tetrahymena thermophila group I intron on the pTT1A3-T7 plasmid (Kind gift of Dr. Thomas R. Cech;⁴⁸). The 9 series αDENV-GrpIs Δ9 and 9v1 as well as the 96 series Δ96 96v1, 96v3, and 96v4, were generated by PCR amplification of the rRNA Tetrahymena thermophila group I intron using the primer sets shown in Additional file 2. Following PCR amplification and band isolation the αDENV-GrpIs were digested with MluI and XhoI and inserted into pA5c-FL. The intron designated 9v1 for the length of its antisense region, was amplified, inserted into pA5c-FL, and named pA5c-9v1.

The 96 series introns were all PCR amplified in two steps. An initial PCR template was created by amplification of pTT1A3-T7 with an initial primer set (see 96 series primer set 1 in ST1) and used as a template for a second round of PCR. For this second PCR step each 96 series was amplified with the same forward primer used in the first PCR step, but with different reverse primers for each 96 series intron (see ST1, “96 series primer set 2”). The resulting introns were named pA5c-96v1, pA5c-96v3, and pA5c-96v4.

ΔP5abc Introns. To create introns missing the P5abc helix, the catalytic core of the intron was first amplified from pTT1A3-T7 and then inserted into the vector pCR2.1-Topo (Invitrogen) to create pCR2.1-GI. Using opposite facing primers 5′ 3′ and with the BsmBI restriction site at each of their 5′ ends, the entire pCR2.1-GI vector containing the intron was amplified, save for the P5abc helix. The resulting PCR product was purified, digested with BsmBI and DpnI and ligated to itself to form the plasmid pCR2.1-ΔP5GI, which contained the catalytic core of the intron without the P5abc helix.

For purposes of creating control vectors, the intron fragments from plasmid pCR2.1-ΔP5GI were amplified with the same primers as the intron inserts from the 9v1, 96v4 series as described above. The products were band isolated and digested with MluI and XhoI, and inserted into the pA5c-FL using the same restriction sites yield pA5c-Δ9v1 and pA5c-Δ96v1, respectively.

Evaluation of αDENV-GrpIs in S2 cells necessitated the construction of double and single-stranded DENV 2-NGC target constructs. The assembly of these is detailed below.

pA5c-EYFP. The DENV 2-NGC target carrier plasmid, pA5c-EYFP, was created by amplification of the EYFP open reading frame from pXL-Bac-EYFP⁴⁹. The The PCR product was band isolated and digested with MluI and XhoI, and inserted into the pA5c-FL plasmid using these same sites.

pA5c D2EYFP single stranded target plasmid. The yeast shuttle vector pRS424-DENV-2 NGC was used as the template for PCR amplification of substrate fragments for the production of the single stranded DENV 2-NGC target substrate. PCR products corresponding to nucleotides 85-267 of the DENV 2-NGC genome were digested with the restriction enzymes BssHII and MluI, and inserted into the MluI digested pA5c-EYFP.

pA5c-D2EYFPD2 double stranded target plasmid. The substrate plasmid bearing the hybridizing sections of the DENV-2 NGC genome at either end of an EYFP open reading frame was made by amplification of the 3′ terminus (nt10495-10723) of the DENV-2 NGC genome from pRS424-DENV-2 NGC. The PCR fragment was digested with the restriction enzymes XhoI and XbaI and inserted into the XhoI, XbaI digested pA5c-D2EYFP plasmid.

pA5c-IRL. The Renilla luciferase normalizing plasmid was created by PCR amplification of the chimeric intron and Renilla luciferase open reading frame from the plasmid pRL-SV40 (Promega). Following band isolation, PCR fragments were digested with XhoI and NotI and inserted into the XhoI and NotI-digested pA5c plasmid.

pA5c-DNA+ctrl. The plasmid directing the constitutive expression of an mRNA corresponding to the predicted trans-spliced product was made by amplification of the DENV-2 fragment from the pA5c-D2-EYFP plasmid followed by digestion of the PCR fragment and the vector pA5c-FL with NotI and XhoI, and ligation of these fragments.

BQCV and DCV-mCherry bearing αDENV-GrpIs

Production of αDENV-GrpI constructs possessing either the BQCV or DCV intergenic IRES sites linked to an mCherry fluorescent marker was achieved through the insertion of PCR amplified BQCV-mCherry or DCV-mCherry fragments into the pA5c-9v1 and pA5c-96v4 plasmids immediately upstream of the 3′ exon, FL (FIG. (FIG. 4a). 4a). The BQCV-mCherry and DCV-mCherry fragments were derived unpublished vectors, pA5c-BQCV-mCherry an pA5c-DCV-mCherry. Prior to insertion PCR amplified BQCV-mCherry and DCV-mCherry fragments as well as the 9v1 and 96v4 bearing constructs were digested with NotI and BamHI. The IRES-linked mCherry PCR fragments were then ligated into the vector constructs.

Reverse transcription-PCR of DENV 2-firefly luciferase splice products derived from cell culture. The total RNA from Dengue virus infected and uninfected cells was extracted using the Qiashredder and RNeasy Mini kits (QIAGEN Inc., Valencia, Calif., USA) in accordance with the manufacturer's instructions and eluted in a final volume of DNAse/RNAse free water to a final volume of 40 μl. The total RNA (5 ug) extracted was treated with 2 U Turbo DNA-free DNAse (Applied Biosystems/Ambion, Inc. Austin, Tex. USA) to rid samples of any DNA contamination, 30 minutes at 37° C. For DNase inactivation, 0.2 volumes of DNase Inactivation Reagent (Applied Biosystems/Ambion, Inc. Austin, Tex. USA) was added to each sample tube and incubated at room temperature for 5 minutes, mixing occasionally. One-step RT-PCR was performed using the SuperScript III One-Step RT-PCR kit (Invitrogen) in accordance with the manufacturer's instructions. cDNA synthesis and PCR amplification were performed as follows: 1) cDNA synthesis at 50° C. for 45 minutes, 2) 40 cycles: denaturation at 95° C. for 2 minutes, annealing at 60° C. for 1 min, and extension at 68° C. for 2 min, 3) final extension of 68° C. for 10 minutes. For detection of the DENV-2 NGC-FL spliced product the forward primer 5′ TCTGATGAATAAC 3′, designed to anneal to DENV2-NGC, and the reverse primer

5′ GAACGTGTACATCGACTGAAATCC 3′,

designed to anneal to FL were used.

Luciferase assays. Schneider 2 (S2) cells were plated into 9.6 cm2 well in minimal S2 media (Gibco) at a density of 1.0 x 106 cells/well. Following the adherence of cells, 3 μg intron, 1 μg double-stranded DENV2 target, and 0.05 μg of IRL expression plasmids were co-transfected into the cells using the Transfectin liposomal transfection reagent (Bio-Rad Laboratories, Hercules, Calif.) in accordance with the manufacturer's protocol. Transfected cell were incubated at 28° C/5% CO2 for 16 hours, washed once in Schneider's media and once in Schneider's media supplemented with 10% FBS and penicillin/strepavidin. These cells were overlaid with Schneider's media supplemented with 10% FBS, 25 μg/mL amphotericin and penicillin/strepavidin, and incubated at 28° C. with 5% CO2 for 72 hours. Following this incubation period cells were gently rinsed twice with 1 ml of 1×PBS pH7.4, and harvested in 300 μl of 1× passive lysis buffer (Promega). The lysates were spun and the supernatants were moved to new tubes. Ten microliters of the supernatant from each tube was added in triplicate wells of a 96 well microtiter plate in and analyzed using the Dual Luciferase System (Promega) with an LMaxII384 Luminometer (Molecular Devices, Sunnyvale, Calif.) with the following parameters: 10 μl of each substrate, 2 second delay, 5 second reading integration. Firefly luciferase readings were normalized against the Renilla luciferase reading by dividing the firefly raw data by the amount of Renilla luciferase detected.

Aag2 cells were plated into 9.6 cm2 well in minimal S2 media (Lonza) at a density of 1.0×10(6) cells/well. At 15 hours post-plating cells were transfected as performed for S2 cells. Following an overnight incubation (16 hours), cells were washed once with 1 ml Schneider's minimal media and once with 1 ml infection media (Schneider's media containing 2% FBS and 1% essential amino acids). Cells were then overlaid with 2 ml infection media containing DENV-2 at an MOI of 0.01, gently rocked for 1 hr to aid in absorption of the virus, then incubated at 28° C. with 5% CO2 for 96 hours. Cells were processed and analyzed for luciferase activity as described for the S2 cells above. All luciferase experiments were performed in triplicate.

TCID50-IFA analysis. Assessment of DENV-2 NGC titre was measured using serial 10 fold dilution followed by detection of the cell surface expressed DENV E protein as previously described⁴. Briefly, cell media containing virus from infected C6/36 cells were accumulated 48 hpi and overlaid onto naive C6/36 cells using 10 fold serial dilutions in a 96 well plate and incubated for 4 days at 28° C. without CO2. Cells were then fixed with acetone:DPBS (3:1) and stained with a primary DENV envelope (E) antibody (1:200)⁵⁰. Positive DENV-2 NGC infected cells were detected using a biotinylated-streptavidin detection system conjugated with Fluorescein isothiocyanate (FITC; Amersham Biosciences, Piscataway, N.J.). Cell cytoplasms displaying fluorescence were scored as positive for DENV infection. The number of positive wells were counted and the virus titers calculated according to Karber's method⁵¹.

Sequence composition of the αDENV-GrpI. The features of each αDENV-GrpI are shown and construction is described in Methods. Right column lists the individual αDENV-GrpIs used in this study. The nucleotide sequences of each region are listed beside the corresponding αDENV-GrpI. Internal guide sequence=IGS, BL=bulge loop, External guide sequence=EGS, P10=P10 helix. (Table 1).

TABLE 1
Sequence File

| αDENV- | | | | |
|---|---|---|---|---|
| GrpIs | IGS | BL | EGS | P10 |
| Δ9 | AUACGGCUU | ACGAGCGUAU | GAGAAACCG | ACUGCUUCG |
| 9v1 | AUACGGCUU | ACGAGCGUAU | GAGAAACCG | ACUGCUUCG |
| Δ96 | UGCGGGAAG | GAGCGAGUUC | 5′ACAGTTTTAATGGTCCTCGTCCCTGCAGCATTCCAAG | ATTCTC |
| | | | TGAGAATCTCTTTGTCAGCTGTTGTACAGTCGACACGC | |
| | | | GGTTTCTGAGCGCTTTCAGCA3′ | |
| 96v1 | UGCGGGAAG | GAGCGAGUUC | 5′ACAGTTTTAATGGTCCTCGTCCCTGCAGCATTCCAAG | ATCCTC |
| | | | TGAGAATCTCTTTGTCAGCTGTTGTACAGTCGACACGC | |
| | | | GGTTTCTGAGCGCTTTCAGCA3′ | |
| 96v3 | UGCGGGAAG | GAGCGAGUUC | 5′ACAGTTTTAATGGTCCTCGTCCCTGCAGCATTCCAAG | ATCCTC |
| | | | TGAGAATCTCTTTGTCAGCTGTTGTACAGTCGACACGC | |
| | | | GGTTTCTGAGCGCTTTCAGCA3′ | |
| 96v4 | UGCGGGAAG | GAGCGAGUUC | 5′ACAGTTTTAATGGTCCTCGTCCCTGCAGCATTCCAAG | ATTCTC |
| | | | TGAGAATCTCTTTGTCAGCTGTTGTACAGTCGACACGC | |
| | | | GGTTTCTGAGCGCTTTCAGCA3′ | |

Primers and PCR fragments. The forward and reverse primer sets used to produce the corresponding PCR fragments are listed. Restriction sites are in lower case text. See Methods for description of vector constructs.

Primers and PCR fragments. All primer sequences are illustrates in the following table. The forward reverse primer sets to produce the corresponding PCR fragments are used. Restriction sites are in lower case text. See methods herein for description of vector constitutes.

TABLE 2
Design Illustration of Anti-Dengue Virus Group 1 Introns

| PCR | |
|---|---|
| Fragments | Forward Primer |
| A5c | 5′CGAggtaccTAAAAAAAATCATGAATGGCAT |
| promoter | CAACTCTG 3′ |
| SV40 late | 5′TGTgcggccgCAGTCTAGAAAAGGATCCTAGATC |
| transcription | ATAATCAGCCATACCACATTTGTAGAGG3′ |
| terminator | |
| and | |
| polyadenylation | |
| signal | |
| pA5c-FL | 5′AAActcgagACCATGGAAGACGCCAAAAACA |
| | TAAAG3′ |
| ΔPabc5 | 5′TTTAAATTTcgtctcCAGACATGGTCCTAACC |
| | ACGCAGCC3′ |
| pA5c-9v1 | 5′AAATCTTacgcgtCGGTTTCTCTATGCGAGCATTCGGC |
| | ATAAAAAGTTATCAGGCATGCACCTGGTAG3′ |
| 96 series | 5′GGGaACGGCTGACACGCGGTTTCTGAGCGCTTTCA |
| primer set 1 | GCACTTGAGCGAGGAAGGGCGTAAAAGTTATCAGGC |
| pA5c-96v1 | ATGCACCTGGTAG3′ |
| pA5c-96v3 | 5′GGGAAAacggctACAGTTTTAATGGTCCTCGTCCCTG |
| pA5c-96v4 | CAGCATTCCAAGTGAGAATCTCTTTGTCAGCTGTTGTAC |
| | AGTCGACACGCGGTTTCTGAGCGC3′ |
| | 5′GGGAAAacggctACAGTTTTAATGGTCCTCGTCCCTG |
| | CAGCATTCCAAGTGAGAATCTCTTTGTCAGCTGTTGTAC |
| | AGTCGACACGCGGTTTCTGAGCGC |
| pA5cEYFP | 5′AAAacggctATGGTGAGCAAGGGCGAGGAGCTG3′ |
| pA5c- | 5′AAAGGGAAAgcgcgcTAATACGACTCACTATAGGGAGTCTAC |
| D2EYFP | GTGGACCGACAAAGACAG3′ |
| pA5c- | 5′TTTCCCTTTctcgagGACTAGCGGTTAGAGGAGACCCC3′ |
| D2EYFPD2 | |
| pA5c-IRL | 5′CCCctcgagGATTCTTCTGACACAACAGTCTCGAACTTAAGC3′ |
| pA5c- | 5′TGTgcggccgcAGTCTAGAAAAGGATCCTAGATCATAATCAGCCA |
| DNA + ctrl | TACCACATTTGTAGAGG3′ |
| BQCV- | 5′CCCGGGCCCgcggccgcTTTTCTTGACTTCTCTTAAAACCAAGAATG3′ |
| mCherry | |

### EXAMPLE 2

### Analysis of Highly Conserved elements in the Dengue genome

All nucleotide position designations used throughout the present disclosure are relative to the published DENV-2 New Guinea strain C genome (DENV-2 NGC; GenBank Accession: M29095). 98 DENV genomes and genome fragments were aligned from the four different serotypes that were present in GenBank using the ClustalX program.

While overall similarity was highest within a given serotype, the alignment showed a significant conserved region between 131 and 164 nt having only one variable base at position 152 nt (FIG. 1a). This sequence was wholly contained within the Capsid (CA) protein gene, and overlapped with the 5′ CS (conserved sequence) identified as essential for replication^{23,25}.

### EXAMPLE 3

### Anti-DENV Group I Trans-Splicing Introns (αDENV-GrpIs) that Target DENV-2 NGC Genomes

Two different uracil bases have been targeted on the positive sense genomic strand within the highly conserved 5′-3′ cyclization sequence (CS) region common to all serotypes of DENV with our αDENV-GrpIs. The preset ribozymes have demonstrated ability to specifically trans-splice a new RNA sequence downstream of the targeted site in vitro and in transfected insect cells as analyzed by firefly luciferase and RT-PCR assays. The effectiveness of these αDENV-GrpIs to target infecting DENV genomes is also validated in transfected or transformed Aedes mosquito cell lines upon infection with unattenuated DENV-2 NGC.

Group I introns were designed to target and catalyze trans-splicing within the conserved sequences of the 5′ CS region of DENV. These introns cleave either single stranded or homologously paired double stranded RNA at defined uracils and covalently join a 3′ exon tag to the end of the cleavage product. These introns were evaluated for activity in both transfected and transformed cell cultures to determine their effectiveness in targeting DENV sequences. Two of these introns, designed 9v1 and 96v4 gave the greatest number of trans-splice product compared to the other Anti-DENV Group I trans-splicing introns (αDENV-GrpI) in each respective series, as judged by luciferase assays. The success of this approach against both subgenomic DENV sequences and infecting DENV genomes provides a potent anti-viral strategy that should prove useful against this important disease.

Analysis shows that the αDENV-GrpIs provided herein have the ability to effectively trans-splice the DENV genome in situ. Notably, these results show that the αDENV-GrpI 9v1, designed to be active against all forms of Dengue virus, effectively targeted the DENV-2 NGC genome in a sequence specific manner. These novel αDENV-GrpI introns provide a striking alternative to other RNA based approaches for the transgenic suppression of DENV in transformed mosquito cells and tissues.

### EXAMPLE 4

### Desing of Anti-DENV Group I Trans-Splicing Introns (αDENV-GrpIs) Targeting Conserved DENV Sequences

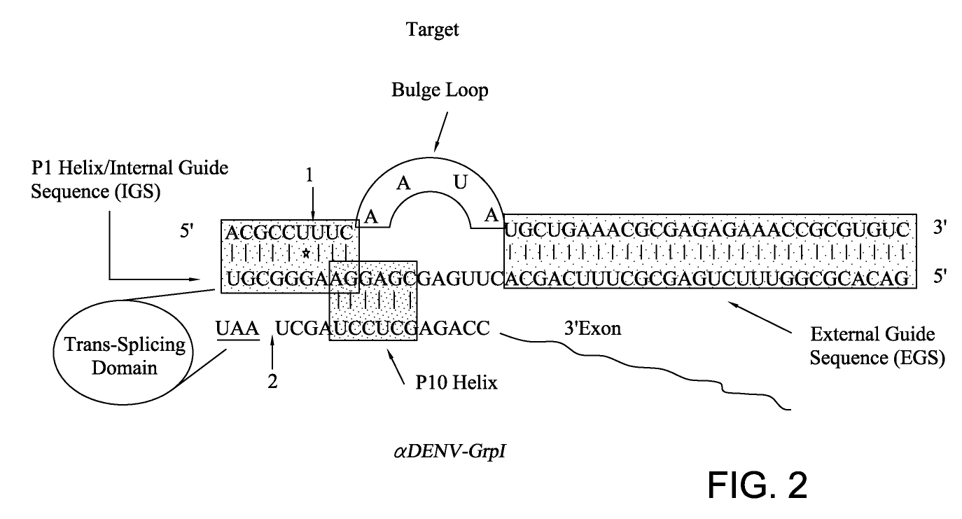

The Group I intron requires an accessible uracil nucleotide downstream of which the target sequence is cleaved. In a trans-splicing reaction, two separate segments of the intron are utilized to specify the RNA sequence the ribozyme targets. The internal guide sequence (IGS), a part of the P1 helix, and external guide sequence (EGS) are each complementary to the target RNA sequence (FIG. 1c step 1 and FIG. 2). The IGS is limited in size to 9 base pairs near the reactive uracil while the EGS can be of any length and forms a transient helix with the target RNA sequence downstream of the reactive uracil²⁰. Biochemical analysis has shown that the activity of Group I introns can be enhanced through 5′- and 3′-splice junction base pairing²⁶.

Trans-splicing group I introns promote the joining of a target sequence to a 3′ exon through two successive yet independent trans-splicing reactions (FIG. 1c). The IGS and EGS of the αDENV-GrpI form Watson-Crick base pairs with the DENV 2-NGC target RNAs. Unintentional expression of the 3′ exon, FL, is prevented by an exogenously inserted UAA stop codon immediately upstream of the UCG splice site. An initial transesterification reaction results in cleavage of the target RNA in a guanoisine dependent manner with conformational change of the EGS. Displacement of the distal portion of the Plhelix by sequences upstream of the 3′ exon forms the P10 helix, allowing a second transesterification reaction to take place. This results in ligation of the DENV 2 NGC genome to the FL 3′exon, promoting firefly expression from the DENV capsid AUG.

While most strategies for identifying optimal IGS sequences utilize a randomized library, called a GN5 library, to locate the most accessible uracil within a given target sequence^{27,28}, the present approach for targeting a specific segment of the DENV genome within the DENV 5′ conserved region limited our choices of uracils, and the GN5 library approach was not an option. A more direct approach for our analyses was therefore utilized.

Anti-DENV Group I trans-splicing introns (αDENV-GrpIs) were designed to target two different uracil bases within the identified conserved region. The first set of introns targeted uracil 143 (U143) and were designed to effectively trans-splice all known DENV sequences (FIG. 1a). All of these introns included a 9 nt antisense External Guide Sequence (EGS) targeting downstream sequences that are also conserved among all DENV genomes to improve the targeting capability of the intron and to minimize potential off-target splicing interactions. A single variable base at nucleotide 152 is positioned within a non-homologous bulge loop structure that separates the IGS and EGS, and therefore does not influence the targeting of the intron (FIG. 2). This bulge loop structure allows the formation of the P10 helix which increases the catalytic efficiency of the intron²⁹.

Intron 9v1 was made with a 9 base P1 helix, and a 9 base EGS (Additional file 1). Excluding the wobble base at position U143 which is required for proper cleavage³⁰⁻³³, 17 bases of this intron interact directly with the intended target sequence.

A second set of αDENV-GrpIs were constructed with an extended 96 base antisense EGS that was engineered to specifically bind to the DENV-2 NGC (FIG. 1b). Each version of this series shared the same EGS and P1 helix, and targeted the same uracil, U132, but differed in their P10 helix. The first version of the 96 series, 96v1, was made with a 6 base pair P10 helix that had no wobble base, and a standard P1 helix of 9 bases, inclusive of the required wobble base (Additional file 1). 96v3 is similar to 96v1 in all respects except the trimming of 3 nucleotides between the P10 helix region and the catalytic core. Finally, version 4 of the 96 series (96v4) incorporated a wobble base pairing downstream of the 3′ exon splice-site. This alteration in the P10 helix has been used in published experiments with other group I introns (9). The 96 series of introns target a different uracil than 9 due to the larger stretch of 100% conserved sequence available for base pairing in the Dengue 2 alignment, thus the reason for the differences in the nucleotide content of the IGS and nucleotide content and length of the EGS sequences.

### EXAMPLE 5

### Assessing αDENV-GrpI Activity by Firefly Lufiferase Assay in S2 Cells

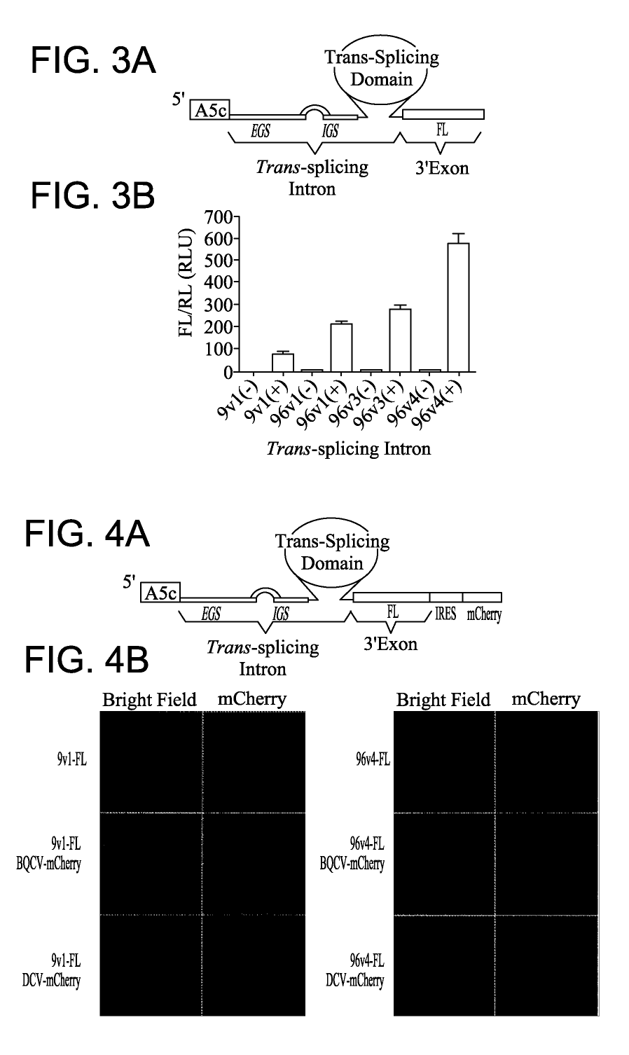

Since each αDENV-GrpI was constructed with a 3′ firefly luciferase (FL) ORF (FIG. 3a), a standard dual luciferase assay was used to assess the ability of each αDENV-GrpI to target the 5′CS region and form a DENV2-FL splice product. The formation of this functional splice product can be used to quantitatively gauge the ability of our anti-DENV group I introns to reprogram a target sequence either in the form of a double-stranded fold back mimic, or in the context of DENV infection.

Drosophila S2 cells were co-transfected with αDENV-GrpI expression plasmids possessing FL as the 3′ exon, dsDENV-2 substrate expression plasmids, and a Renilla luciferase expression plasmid, pA5c-IRL, to normalize the readings (FIG. 3b). As a negative control, cells were co-transfected with the pUC57 empty vector as a substitute for the substrate, at the same concentration as the dsDENV-2 concentration used in the experimentals. S2 cells were harvested 48 hours post-transfection, processed and analyzed as described in Methods. All introns demonstrated firefly luciferase activity, and therefore successful targeting of the pA5c-D2-EYFP-D2 construct. αDENV-GrpI 96v4 produced the greatest amount of luciferase activity, and therefore the greatest amount of trans-spliced product, of all the 96 intron series studied (FIG. 3b). The αDENV-GrpI as9v1, designed to target all DENV serotypes, also produced significant FL activity verifying its ability to attack our DENV mimic in these cells.

### EXAMPLE 6

### Engineering and Assessment of Bicistronic αDENV-GrpI 9v1 and 96v4 Intron Constructs in Drosophila S2 Cells

Since αDENV-GrpIs 9v1 and 96v4 were determined to be the best candidate introns of each series by the in vitro assay we assessed the activities of these introns in transfected cell culture assays. Each of the introns was tagged downstream of the 3′ exon with the mCherry fluorescent marker gene expressed from an IRES sequence of either the Black queen cell virus (BQCV) or Drosophila C virus (DCV) (FIG. 4a). These Dicistrovirus IRES sequences were previously determined to yield the highest levels of expression in Ae aegypti mosquito and D. melanogaster S2 cells³⁴. This This bi-cistronic configuration allowed monitoring for the presence and expression of the αDENV-GrpI constructs within cell cultures. As expected, IRES-mediated expression of the mCherry fluorescent marker occurred upon transfection of these bi-cistronic constructs in S2 cells (FIG. 4b.).

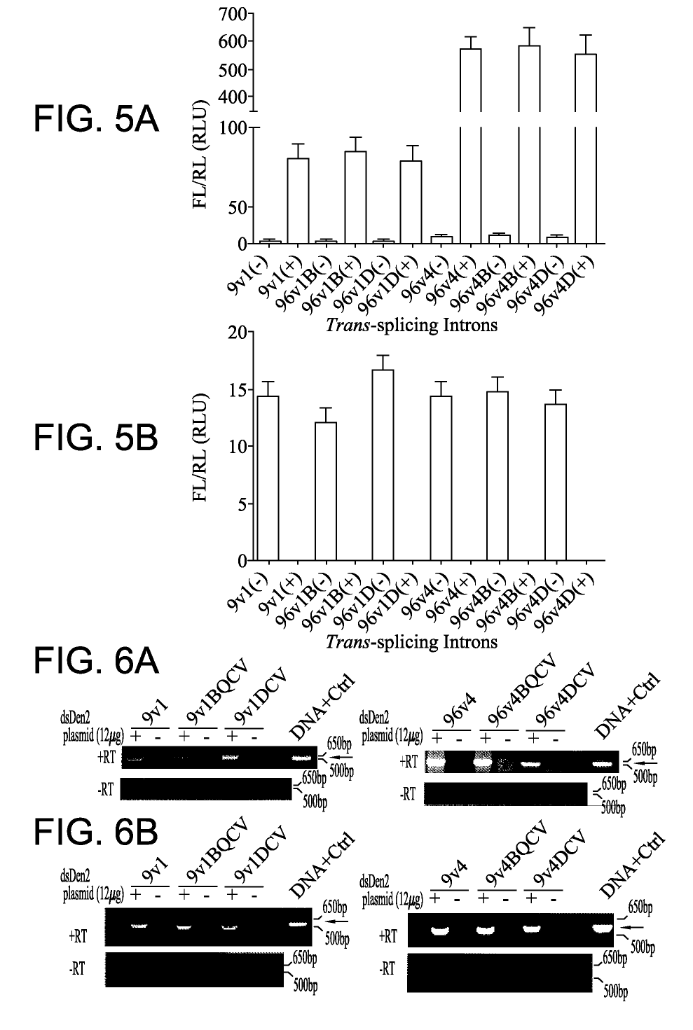

The influence of the addition of the IRES-mCherry configuration on αDENV-GrpI activity was examined by performing dual luciferase assays (see Methods) with the bicistronic αDENV-GrpIs 9v1 and 96v4 intron constructs in transient transfected cell culture (FIG. 5a). D. melanogaster S2 cells were transiently transfected with αDENV GrpI 9v1 or 96v4 either unlinked or linked to an IRES/mCherry driver, and were challenged with a double-stranded fold back construct designed to mimic the 5′-3′ CS region of DENV-2 (dsDENV-2; see Methods) in the presence of the pA5c-IRL normalizer. To rule out non-specific targeting of the anti-DENV introns to cellular targets, control cells were transfected with the pUC57 plasmid in lieu of the dsDENV-2 construct.

FL activity was greatest for the 96v4 IRES-mCherry-linked or unlinked constructs in S2 cells (Figure (FIG. 5a), with no statistical differences in activities among the 96v4 intron constructs. This confirms that addition of the 3′ IRES/mCherry configuration does not alter the trans-splicing capabilities of the 96v4 intron, since equivalent FL counts were obtained for both 96v4 and 96v4 IRES/mCherry constructs.

Similarly, the overall activities of the 9v1 intron constructs, whether IRES-mCherry linked or unlinked, were statistically similar. However, the overall levels of activation were substantially lower than those detected in cells expressing 96v4 introns, possibly due to the shorter EGS⁷ target accessibility leading to a decrease in the production of trans-spliced product.

αDENV-GrpIs 9v1 or 96v4, IRES-mCherry linked or unlinked, were either transiently or stably expressed in S2 cells, and analyzed by RT-PCR 72 hours post-transfection with the dsDENV-2 target plasmid using heterologous primers (see Methods). Splice product bands were excised, gel purified, and sequenced to confirm their identity. The specific DENV-FL splice product was detected by RT-PCR in transfections with both αDENV-GrpI 9v1 and 96v4 in S2 cells as evidenced by the presence of a 580 by band, no splice product was detected in the absence of the target dsDENV-2 expression plasmid (FIGS. 6a and 6b).

### EXAMPLE 7

### αDENV-GrpIs Effectively Target DENV-2 NGC in Mosquito Cells

The effectiveness of the αDENV-GrpI introns to target infecting DENV genomes was assessed by FL assays following DENV-2 challenge of Ae. aegypti Aag2 cells transiently transfected with αDENV-GrpI introns (FIG. (FIG. 5b). 5b). αDENV-GrpI and pA5c-IRL expression plasmids were co-transfected into Aag2 cells, and were challenged with DENV-2 NGC at an MOI of 0.01 24 h post transfection. Control cells were transfected with an empty pUC57 plasmid, in place of the plasmids and challenged with virus in the same manner. Each of the αDENV-GrpIs displayed levels of FL activity indicating successful splicing against the infecting DENV. In this case FL activity was only slightly greater for the 96v4 IRES-mCherry-linked or unlinked constructs in Aag2 cells, with no statistical differences in activities among the 96v4 intron constructs. Similarly, the overall activities of the 9v1 intron constructs, whether IRES-mCherry linked or unlinked, were statistically similar. As seen in the previous assays, the overall levels of activation were somewhat lower than those detected in cells expressing 96v4 introns most likely due to the shorter EGS⁷ or target accessibility. This confirmed the activity of our αDENV-GrpIs against actual infecting virus, and demonstrated that addition of the 3′ IRES/mCherry configuration does not appear to alter the ability of the αDENV-GrpIs tested to target DENV genomes in cells. The overall levels of luciferase activity were lower in these virally infected mosquito cells than those observed in S2 cells transfected with a plasmid construct expressing an artificial target sequence. This may be due to the role viral infection plays in host cell RNA and protein expression, or may be due to potential basic differences in nascent RNA and protein expression between these two cell lines.

Transient transfection of 9v1, 96v4 and inactive ribozymes Δ9 and Δ96 was performed in C6/36 cells followed by RT-PCR analysis to confirm the detection of splice product (FIG. 7a). No splice product was observed in the presence of the inactive ribozymes Δ9 and Δ96 showing that the splice product detected is due to the trans-splicing activities of the αDENV-GrpIs.

### EXAMPLE 8

### αDENV-GrpIs 9v1 and 96v4 Effectively Target DENV Genomes in Transiently Transfected Cells

The activities of the αDENV-GrpI introns in transformed mosquito cell culture assays were assessed (FIG. 7b). To produce Ae. albopictus C6/36 cells transformed with each bicistronic αDENV-GrpI intron construct, cells were co-transfected with each αDENV-GrpI construct and a plasmid possessing the hygromycin resistance gene. Transfection media was replaced with selective media at 48 hours post transfection. Cells were then passaged several times per week in selection media. The concentration of hygromycin used was increased with each passage until a final concentration of 10 mg/ml was reached. mCherry fluorescence and RT-PCR were used to confirm expression of the introns in the transformed cultures.

αDENV-GrpIs 9v1 and 96v4 linked to either the BQCV or DCV IRES elements expressing mCherry were stably expressed in Ae. albopictus C6/36 cells and challenged with DENV-2 NGC at an MOI of 0.1 at 24 h post transfection. Control cells were transfected with an empty pUC57 plasmid and challenged with virus in the same way (FIG. 7b).

Cells were processed and analyzed by RT-PCR 4 days post-infection with heterologous primers to detect the DENV-FL splice product, and identified bands were excised, gel purified, and sequenced to confirm their identity. DENV-2-FL splice product was detected in C6/36 cells when introns were expressed in a transformed cell manner, and whether the intron was linked with either IRES-mCherry configuration (FIG. 7b). No control DENV-2-FL splice product was detected by RT-PCR in cells transfected with the pUC57 control vector. Significantly, these results also show that the 9v1 intron, designed to be active against all forms of Dengue virus, is capable of effectively targeting the DENV 2-NGC genome in a sequence specific manner.

### EXAMPLE 9

### Expression of αDENV-GrpIs 9v1 and 96v4 in Mosquito Cells Leads to Suppression of DENV-2 NGC

The final step in the present analysis of the αDENV-GrpI intron constructs was to determine their ability to suppress overall infectious DENV-2 NGC production in cell culture using tissue culture infectious dose immunofluorescence antibody (TCID50-IFA) assays (FIG. 7c;⁴). αDENV-GrpI-FL constructs were stably expressed in C6/36 cells, challenged with DENV-2 NGC, and assayed as described above.

αDENV-GrpIC6/36 cell lines 9v1 and 96 v4 displayed vast reductions in viral titer, up to 3 log, when compared to the infection control (I). Suppression of virus replication is evident regardless of whether the intron expressed in the cells was the 96v4 trans-splicing intron, engineered to specifically target DENV-2, or the 9v1 trans-splicing intron, which was designed to target all Dengue virus serotypes. This anti-viral effect was independent of the IRES-mCherry configuration used in the anti-DENV constructs. Though the 96v4 intron appeared to suppress DENV-2 NGC replication to a greater extent than 9v1, a direct comparison of activities cannot be considered valid since αDENV-GrpIs 9v1 and 96v4 target different uracils (4).

### EXAMPLE 10

### Structure and Activity of αDENV-Grp1 Introns

Any approach that inhibits virus infection by direct interaction with the RNA genome or expressed mRNAs must be designed to act upon invariant sequences to be optimally effective. Since the DENV genome is subject to great variation throughout most of its sequence, an anti-DENV Group I trans-splicing introns (αDENV-Grp1) was designed to target and catalyze trans-splicing within the highly conserved 5′ Circularization Sequence (CS) region of the DENV genome (ref.). This sequence is important for replication of the virus genome through the formation of a panhandle structure upon association with the 3′ CS sequence, constraining its variability.

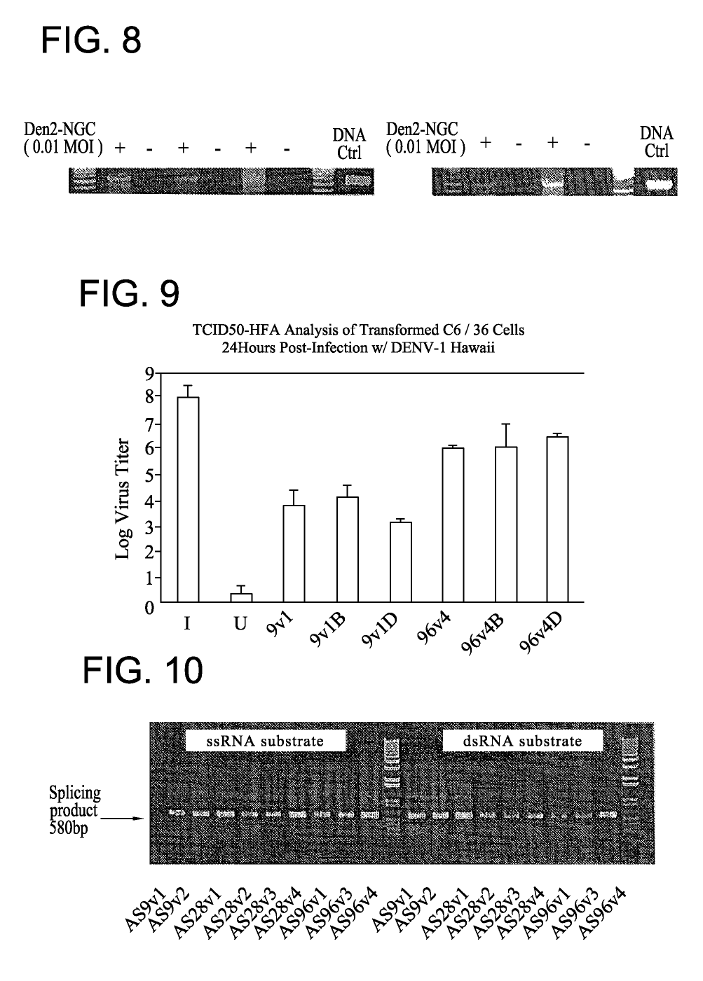

Introns were expressed using either an in vivo transcription system or within mosquito cells and combined with an expressed target RNA molecule composed of the 5′ terminal 450 nt of the DENV genome in which the target sequence resides (FIG. 2) or with infecting DENV (FIG. 8). Splice products were detected by RT-PCR for the single-stranded RNA target, and for double stranded RNA target designed to mimic the panhandle structure formed by the 5′ CS and 3′ CS association (FIG. 10). Splice product was also detected in mosquito cells transiently transformed and challenged with virus (FIG. 3).

### EXAMPLE 11

### Apoptosis-Inducing 3′ Exon

While the αDENV-Grp1 may successfully repress viral infection by physically attacking the invading DENV genome upon initiation of infection, it was considered that if replication initiated, there would be a possibility for the virus to overcome the activity of the intron through sheer numbers, in the absence of a more potent induction of cell death. Taking advantage of the splicing capabilities of the Group I intron, a 3′ exon was designed that would encode a potent apoptotic inducing product, tBax.

In a healthy non-apoptotic cell, the full-length form of tBax, Bax, is held in dynamic equilibrium with anti-apoptotic protein, BCL-2, in the form of a heterodimer (ref.). Activation of apoptosis through the BCL-2 pathway, dephosphorylates Bad to have a high affinity for BCL-2 and sequesters BCL-2 away from Bax, leading to an overall increase in the levels of free Bax in the cell. Once free Bax reaches a certain threshold, it is cleaved by native cofactors in the cell into its active form. tBax, which multimerizes and embeds in the outer membrane of the mitochondria, causing membrane depolarization and formation of large pores. Cytochrome C spills out of the mitochondria, setting off a cascade of apoptotic effects, committing the cell to apoptosis.

tBax exhibits a number of appealing characteristics as an ideal choice for the selected pro-apoptotic gene of the present methods. First, it acts alone, requiring no native cofactors to form a pore and kill the cell. Next, tBax-induced apoptosis is unrescuable, native anti-apoptotic factors are not stimulated by the presence of exogenous tBax. Once Cytochrome C is released from the mitochondria, the cell initializes its own set of pro-apoptotic factors to ensure that it is killed in an ordered and non-inflammatory fashion. Finally, tBax has successfully killed all cells in which it has been expressed.

The effectiveness of the presently described αDENV-Grp1-tBax introns against all serotypes and strains of DENV is illustrated in FIG. 9. By selecting the conserved CS sequence, a remarkably effective targeting of the conserved domain among all serotypes of DENV was achieved. The best results were obtained with the 9 version, which targets 17 nt of sequence absolutely conserved among all DENV genomes. The suppression of virus titers in cells expressing these introns was significant, and in the case of the 9 version introns, effectively achieved 4 to 5 logs.

While not intending to be limited to any particular mechanism of action or theory, it is believed that at least one explanation for the background levels of infection in these cultures may at least in part be the result of non-transformed hygromycin resistant cells persisting in the cultures.

All patents, publications and abstracts cited herein are incorporated herein by reference in their entirely. It should be understood that the foregoing relates only to certain embodiments of the present invention, and that numerous modifications or alterations may be made therein without departing from the spirit and the scope of the present invention as defined in the following claims.

### Bibliography

The following references are specifically incorporated herein in their entirety.

1. Clyde K, et al., (2006), Virol. 80 (23):11418-11431. doi: 10.1128/JVI.01257-06.

2. James A A., (2005), Trends Parasitol. 21 (2):64-67. doi: 10.1016/j.pt.2004.11.004.

3. Sinkins S P, Gould F., (2006), Nat Rev Genet. 7 (6):427-435. doi: 10.1038/nrg1870.

4. Nawtaisong P, Keith J, et al., (2009), Virol J. 6:73. doi: 10.1186/1743-422X-6-73.

5. Cech T R., (1991), Cell. 64 (4):667-669. doi: 10.1016/0092-8674(91)90494-J

6. Long M B, et al., (2003), J Clin Invest. 112 (3):312-318

7. Ayre B G, (1999), Proc Natl Acad Sci USA. 96 (7):3507-3512. doi: 10.1073/pnas.96.7.3507.

8. Byun J, et al., (2003), Rna. 9 (10):1254-1263. doi: 10.1261/rna.5450203.

9. Jung H S, et al., (2005), Biotechnol Lett. 27 (8):567-574. doi: 10.1007/s10529-005-2883-6.

10. Kastanos E, et al., (2004), Biochem Biophys Res Commun. 322 (3):930-934. doi: 10.1016/j.bbrc.2004.07.203.

11. Waterston R H, et al., (2002), Nature. 420 (6915):520-562. doi: 10.1038/nature01262.

12. Ryu K J, Lee S W., (2003), J Biochem Mol Biol. 36 (6):538-544.

13. Ryu K J, Lee S W., (2004), J Microbiol. 42 (4):361-364.

14. Sullenger B A, Cech T R., (1994), Nature. 371 (6498):619-622. doi: 10.1038/371619a0.

15. Lander E S, et al., (2001), Nature. 409 (6822):860-921. doi: 10.1038/35057062.

16. Jeong J S, et al., (2008), Clin Cancer Res. 14 (1):281-290. doi: 10.1158/1078-0432.CCR-07-1524.

17. Kwon B S, et al., (2005), Mol Ther. 12 (5):824-834. doi: 10.1016/j.ymthe.2005.06.096.

18. Watanabe T, Sullenger B A., (2000), Proc Natl Acad Sci USA. 97 (15):8490-8494. doi: 10.1073/pnas.150104097.

19. Rogers C S, et al., (2002), J Clin Invest. 110 (12):1783-1789.

20. Kohler U, et al., (1999), J Mol Biol. 285 (5):1935-1950. doi: 10.1006/jmbi.1998.2447.

21. Ryu K J, et al., (2003), Mol Ther. 7 (3):386-395. doi: 10.1016/S1525-0016(02)00063-1.

22. Hahn C S, et al., (1987), J Mol Biol. 198 (1):33-41. doi: 10.1016/0022-2836 (87)90455-4.

23. Alvarez D E, et al., (2005), J Virol. 79 (11):6631-6643. doi: 10.1128/JVI.79.11.6631-6643.2005.

24. Markoff L., (2003), Adv Virus Res. 59:177-228.

25. Alvarez D E, et al., (2008), Virology. 375 (1):223-235. doi: 10.1016/j.viro1.2008.01.014.

26. Burke J M., (1989), FEBS Lett. 250 (2):129-133. doi: 10.1016/0014-5793(89)80704-5.

27. Jones J T, et al., (1996), Nat Med. 2 (6):643-648. doi: 10.1038/nm0696-643.

28. Lan N, et al., (1998), Science. 280 (5369):1593-1596. doi: 10.1126/science.280.5369.1593.

29. Bell M A, et al., (2004), Biochemistry. 43 (14):4323-4331. doi: 10.1021/bi035874n.

30. Cech T R., (1990), Annu Rev Biochem. 59:543-568. doi: 10.1146/annurev.bi.59.070190.002551.

31. Strobel S A, Cech T R., (1993), Biochemistry. 32 (49):13593-13604. doi: 10.1021/bi00212a027.

32. Campbell TB, Cech TR., (1996), Biochemistry. 35 (35):11493-11502. doi: 10.1021/bi960510z.

33. Guo F, Cech T R., (2002), RNA. 8 (5):647-658. doi: 10.1017/S1355838202029011.

34. Carter J R, et al., (2008), J Gen Virol. 89 (Pt 12):3150-3155. doi: 10.1099/vir.0.2008/003921-0.

35. Peng T, et al., (2009), Can J Microbiol. 55 (2):139-145. doi: 10.1139/W08-107.

36. Acosta E G, et al., (2008), J Gen Virol. 89 (Pt 2):474-484. doi: 10.1099/vir.0.83357-0.

37. Caplen N J, et al., (2002), Mol Ther. 6 (2):243-251. doi: 10.1006/mthe.2002.0652.

38. Adelman Z N, et al., (2001), Insect Mol Biol. 10 (3):265-273. doi: 10.1046/j.1365-2583.2001.00267.x.

39. Adelman Z N, et al., (2002), J Virol. 76 (24):12925-12933. doi: 10.1128/JVI.76.24.12925-12933.2002.

40. Adelman Z N, et al., (2004), Transgenic Res. 13 (5):411-425. doi: 10.1007/s11248-004-6067-2.

41. Franz A W, et al., (2006), Proc Natl Acad Sci USA. 103 (11):4198-4203. doi: 10.1073/pnas.0600479103.

42. Olson K E, et al., (1996), Science. 272 (5263):884-886. doi: 10.1126/science.272.5263.884.

43. Du Q, et al., (2005), Nucleic Acids Res. 33 (5):1671-1677. doi: 10.1093/nar/gki312.

44. Ng C Y, et al., (2007), Antiviral Res. 76 (3):222-231. doi: 10.1016/j.antivira1.2007.06.007.

45. Zhang W, et al., (2004), Genet Vaccines Ther. 2 (1):8. doi: 10.1186/1479-0556-2-8.

46. Thompson J D, et al., (1994), Nucleic Acids Res. 22 (22):4673-4680. doi: 10.1093/nar/22.22.4673.

47. Pinkerton A C, et al., (2000), Insect Mol Biol. 9 (1):1-10. doi: 10.1046/j.1365-2583.2000.00133.x.

48. Joyce G F, Inoue T., (1989), Nucleic Acids Res. 17 (2):711-722. doi:

10.1093/nar/17.12.4885.

49. Li X, Lobo N, et al., (2001), Mol Genet Genomics. 266 (2):190-198. doi: 10.1007/s004380100525.

50. Henchal E A, et al., (1985), Am J Trop Med Hyg. 34 (1):162-169.

51. Karber G., (1931), Arch Exp Pathol Pharmk. 162:480-483. doi: 10.1007/BF01863914.

52. Johnson T H, et al., (2005), Proc Natl Acad Sci USA. 102 (29):10176-10181. doi: 10.1073/pnas.0501498102.

What is claimed is:

1. A method for inhibiting mosquito transmission of Dengue virus infection to an animal, said method comprising:
exposing a population of mosquito cells to a ribozyme effector gene to provide a population of transformed mosquito cells, said ribozyme effector gene comprising a trans-splicing intron that specifically targets a uracil residue within a capsid encoding sequence of a Dengue virus genome, said ribozyme having a conserved Dengue virus sequence corresponding to a Dengue virus capsid encoding sequence, wherein transformed mosquitoes are resistant to infection by Dengue virus; and
inhibiting the transmission of the Dengue virus in the transformed mosquito population.

2. The method of claim 1 wherein the conserved Dengue capsid encoding sequence is a native Dengue virus capsid conserved sequence C131 to G151, said ribozyme targeting a uracil residue at native Dengue virus capsid sequence at position 143, position 132, or both a uracil at position 132 and position 143.

3. The method of claim 1 wherein the intron is an anti-DENV Group I trans-splicing intron (αDENV-GrpI).

4. The method of claim 1 wherein the animal is a human.

5. The method of claim 3 wherein the anti-DENV Group I trans-splicing intron (αDENV-GrpI) is αDENV-GrpI 9v1 or αDENV-GrpI 96v4.

6. An αDENV-GrpI intron genetic construct for use in the transformation of a mosquito comprising:
(a) an isolated Dengue virus capsid nucleic acid sequence that is complementary to a native target Dengue virus CS nucleotide sequence from nucleotide position 131 to nucleotide position 151;
(b) an Internal Guide Sequence (IGS); and
(c) an External Guide Sequence (EGS),
wherein said IGS is located 9 base pairs from a reactive uracil (U) target nucleic acid in a Dengue virus genome, and said IGS is complementary to a PI helix.

7. The αDENV-GrpI intron genetic construct of claim 6 when said EGS is capable of forming a transient helix with a target RNA sequence located downstream of a reactive uracil (U) residue within said native target Dengue virus CS nucleotide sequence.

8. The αDENV-GrpI intron genetic construct of claim 6 when said reactive uracil (U) residue is a uracil residue located at position 143, position 132, or both position 132 and position 143.

9. The αDENV-GrpI intron genetic construct of claim 6 wherein said the anti-DENV Group I trans-splicing intron (αDENV-GrpI) is αDENV-GrpI 9v1 or αDENV-GrpI 96v4.

10. A population of insect cells enriched for transformed insect cells resistant to infection by Dengue virus, said transformed insect cells being transformed with the αDENV-GrpI intron genetic construct of claim 6.

11. The population of insect cells of claim 10 wherein said transformed insect cells are mosquito cells.
